(19) United States
(12) Patent Application Publication
(10) Pub. No.: US 2011/0166631 A1
(43) Pub. Date: Jul. 7, 2011

(54) LUMINESCENT CYCLE REGULATOR AND FERTILITY INDICATOR

(76) Inventor: Peter M. Breining, San Mateo, CA (US)

(21) Appl. No.: 12/737,372

(22) PCT Filed: Jul. 7, 2009
(86) PCT No.: PCT/US2009/003961

§ 371 Date: Mar. 25, 2011

Related U.S. Application Data
(60) Provisional application No. 61/134,452, filed on Jul. 10, 2008.

(51) Int. Cl. A61N 5/06

(52) U.S. Cl. 607/88

(57) ABSTRACT

A natural family planning device (10) and method which improves the regularity of the female cycle and indicates days during the cycle when there is an increased probability of conception during intercourse. The device (10) provides automated light therapy regimens of varying intensity and duration while the user sleeps with the aim of synchronizing the user's cycle to the illumination cycle provided by the device thereby improving the regularity of the user's cycle. The device also indicates (50, 55) to the user which days of the synchronized cycle represent the highest likelihood of conception during intercourse. This enables the user to time intercourse to coincide with or avoid the dates of increased probability of conception depending on the desire of the user to become or avoid becoming pregnant.

### CROSS REFERENCE TO RELATED APPLICATIONS

This application claims priority from a U.S. Provisional Application No. 61/134,452 filed on 10 Jul. 2008 by Peter Breining. The entire contents of that application are incorporated by reference herein.

### TECHNICAL FIELD

The present invention relates generally to natural family planning devices and methods, and more particularly to devices that improve the regularity of the female cycle so that intercourse can be synchronized to dates of high or low probability of conception, depending on the user's desire to become or avoid becoming pregnant.

### BACKGROUND ART

The ability to control through natural means the probability of conception during intercourse has long been an attractive goal to many couples. While contraception or conception are both achievable through artificial means such as oral contraceptives and in vitro fertilization respectively, many couples do not avail themselves of these measures because of either moral objection, the required expense, or the intrusiveness and perceived artificiality of the methods. Those couples who do not use these artificial means are left with few options when it comes to reliably influencing the probability of conception through natural means.

The first and simplest method consists of tracking the monthly cycle of the woman and attempting to predict when ovulation will occur so that intercourse can be timed to account for the fact that conception is most likely to occur when intercourse takes place near ovulation. Those attempting to become pregnant will purposefully engage in intercourse near the dates of expected ovulation and those attempting to avoid becoming pregnant will purposefully avoid intercourse near the expected dates of ovulation. This technique, sometimes referred to as the rhythm method, suffers from several shortcomings. First there is the administrative issue of efficiently and repeatably tracking the onset and duration of the woman's cycle. Devices for performing this activity include everything from simple annotations of a calendar, to moveable beads on a necklace, to digital devices such as smart-phones and personal data assistants. Whatever device is used, they all rely on the user to accurately record the start and stop date of her cycle. Failure to enter the data accurately makes the technique less reliable.

Even given reliable data entry these tracking systems cannot overcome the second weakness of this approach which is the variability in cycle duration exhibited in many women. While the theoretical duration of a woman's menstrual cycle is 28.5 days, many women undergo cycles that range in duration from 25 to 45 days. Moreover, the same women can experience dramatic variations in the length of their cycle from one month to the next. Consequently, the reliability of predicting ovulation for women with highly variable cycles can be quite low. This variability can make the usefulness of simply tracking a woman's cycle of little value as a means of natural family planning.

The second method available to those seeking a natural family planning system can broadly be labeled as tracking plus detection. This method employs the tracking procedure as described earlier and combines it with a positive means of detecting ovulation in a woman such as, use of basal thermometers to detect body temperature changes that are often present during ovulation, use of pH measurement devices to detect changes in acidity in the women's saliva, and inspection of vaginal mucous viscosity. The shortcoming of these methods is that while they may in fact provide some indication of ovulation for some women, the information they provide may come too late in that the highest probability of conception results from intercourse that occurs 1-3 days before ovulation, not during ovulation. Furthermore, all of these techniques suffer from a certain intrusiveness and many women complain that the need to routinely implement these measures interferes with the spontaneity and enjoyment of intercourse, precisely one of the things that many who choose natural family planning methods are hoping to preserve.

The third approach available to those seeking a natural family planning system is directed at improving the regularity of the women's cycle so that predictions of when ovulation will occur can be made with improved accuracy and reliability. Improving the regularity of a woman's cycle can be achieved with hormonal and chemical agents, as shown in such references as U.S. Pat. No. 5,721,278 and U.S. Pat. No. 5,589,457. This or similar treatments may take the form of a daily pill ingested by the female, as well as other means of delivering the treatment, such as transdermal patches, implants or oral dosages with frequencies other than daily. The objective of these therapies is to alter the women's hormone levels in order to improve the regularity and predictability of her cycle. These drug therapies usually require physician's prescription and incur an ongoing cost in that the therapies must be continued from month to month to remain effective. Aside from the ongoing cost associated with drug therapy, this approach also has the possible drawback of causing health side-effects in the woman. These side-effects can range from mild effects, such as mood changes and weight gain, to more severe effects such as loss of bone density and potentially increased likelihood of breast cancer. Furthermore, these approaches are generally regarded as too artificial by those who chose natural family planning in that they are only slightly different than using oral contraceptives, which themselves are primarily made of hormones.

Other approaches to improving the regularity of the female cycle include the use of light therapy whereby the woman is periodically exposed to dosages of light in an effort to synchronize, or phase lock, the women's cycle to the schedule of the light exposure, so that her cycle becomes more regular and ovulation can be predicted with greater certainty. The science of using illumination cycles to control female cycle regularity is well established. Scientists have long speculated that there is a relationship between the 29.5 day synodic lunar cycle and the approximately 28 day female menstrual cycle. As modern living and artificial lighting reduced the exposure of the typical woman to the synchronizing effects of the lunar cycle, the awareness of this link and the regularity of some women's cycles decreased. Research in the 1960s and 1970s re-established this somewhat forgotten association between the lunar and female menstrual cycle and documented the association in several publications including: M A Menkin, “On the possibility of a Perfect Rhythm Method of Birth Control by Periodic Light Stimulation,” 99 American Journal of Obstetrics and Gynecology, 1016 (1967); and Dewan, “Effect of Photic Stimulation on the Human Menstrual Cycle,” 27 Photochemistry and Photobiology, 581 (1978); as well as Daniel F. Kripke, “Light Regulation of the Menstrual Cycle,” in “Light and Biological Rhythms in Man,” (L. Wetterberg ed. Pergamon Press 1993).

U.S. Pat. No. 5,589,741 describes a device that makes use of the synchronizing effect of periodic light exposure on a woman's cycle but does not allow for the programming of the light therapy to correspond with the start of the user's cycle, nor does it provide indicating means to inform the user what day of her cycle she is in and whether that day presents an increased or decreased likelihood of conception during intercourse.

U.S. Pat. No. 6,497,718 describes a method for exposing a women to nightly light treatments of varying duration during her sleep. The treatments are intended to roughly mimic the exposure provided by the middle nights of a lunar cycle. However, the invention provides no device for creating the simulated moonlight, lacks any automated method of delivering the therapy, does not propose any other intensity, duration or schedule other than the exact replication of moonlight, and provides no method for indicating to the user which day of her cycle she is in and whether that day presents an elevated probability of conception.

Therefore, a device and method for improving the regularity of a female's cycle and a means of predicting and indicating which dates of the current cycle represent the highest probability of conception without requiring intrusive, artificial, drug-based or medically intensive means of achieving the enhanced cycle regularity and prediction accuracy would be a desirable improvement in the art. Other advantageous improvements could include devices that do not interfere with the spontaneity of intercourse by requiring pre-intercourse diagnostic home-testing. An ovulation cycle regulation device and method which naturally and unobtrusively improves the regularity of a woman's cycle and more accurately predicts and indicates which dates of a woman's cycle present an increased likelihood of conception so that intercourse can be synchronized to dates of high or low probability of conception, depending on the user's desire to become pregnant or to avoid becoming pregnant, would also be a desirable improvement in the art.

### DISCLOSURE OF INVENTION

The present disclosure advantageously addresses one or more of the aforementioned deficiencies in the field of natural female cycle regulation by providing a female cycle regulation device that does at least one of the following: synchronizes the female cycle to a predictable schedule; informs the user during which days of her cycle intercourse is mostly likely to result in conception without the need for artificial, obtrusive or invasive methods to improve cycle regularity or prediction accuracy.

An embodiment of the present disclosure comprises a luminescent female cycle regulator that emits light in a varying range of intensities, colors and duration in order to expose a woman to a dosage of said light during sleep or daytime treatment sessions with the aim of entraining, or phase locking, the women's cycle to match the illumination cycle simulated by the device. By repeatedly exposing the woman to this regimen of light treatment, her irregular menstrual cycle becomes entrained to the simulated illumination cycle created by the device, thereby allowing her to synchronize the timing of intercourse with those dates of her cycle that offer higher or lower probabilities of conception depending on whether her goal is to increase or decrease the odds of conception. After exposure to the monthly treatment regimen, the woman's cycle can be entrained to a cycle similar in duration to that specified by the device, making the prediction by the device of which dates of the cycle represent heightened probabilities of conception during intercourse more accurate.

An embodiment of the luminescent female cycle regulator can be small enough to be placed on a bedside nightstand and comprises a housing that holds a light source for producing the light and a controller, connected to the light source by circuitry, for calculating and controlling the dosage and schedule of the light therapy. The controller can also be connected by circuitry to input controls on the surface of the housing that are employed by the user to indicate the start date of the menstrual cycle and the time at which the user goes to bed each night. Indicator lights can be arrayed on and inside the housing and can be connected to the controller through circuitry and indicate to the user which day of her cycle she is in and whether intercourse on that day would result in an increased likelihood of conception. An AC power cord attaches to the unit and supplies it with electrical power.

Another embodiment of the device is configured as a visor, and can be supplied by DC batteries so that it can be worn by the user during treatment without exposing other people who might share the user's room to the light therapy. A light source can be positioned in the underside of the visor while the controller, input devices, indicator lights, DC power source and circuitry all are positioned on the top side of the visor.

In an embodiment the light source is designed to produce light in a wide range of colors (temperatures) and intensities, including but not limited to, that of natural moonlight.

In an embodiment the user interface can be a simple two-button control system whereby the user presses one button at the start of her cycle and actuates a second button upon going to bed each night. By actuating the second control every night upon going to bed the user delays the start of the nightly therapy session by a specified period of time, thereby enabling her to fall asleep before the light treatment starts.

In yet another embodiment the device can also comprise a reflector element surrounding the light source so that light from this source is directed upward toward the ceiling of the room and away from the eyes of the user so as not to disturb her during sleep.

In an embodiment the device can also comprise a light sensor so that the controller can further modulate the intensity of the simulated illumination based on how much of the light is being reflected off the ceiling and walls of the room in which it is being used. This can allow the device to emit greater light, in darkly colored, poor reflectance, larger rooms and less light in small, brightly colored, high reflectance rooms, thereby ensuring that the prescribed dosage is consistently delivered in different usage environments.

In an embodiment there can be an indicator light for every day of a 29 day cycle. The indicator light corresponding to each day of the cycle lights up in response to a signal from the controller that contains a clock that is triggered when the user activates an input device on the first day of her period. The indicator lights corresponding to the dates when there is a heightened probability of conception during intercourse can be distinguishable from the remaining lights.

In still another embodiment the means of indicating that the women is entering a period of heightened probability of conception during intercourse can be indicated by a change in color of the entire upward-facing, top shell of the unit.

In still another embodiment, the device can incorporate a red-light user activated nightlight that enables the user to see during the night without significantly negatively affecting the nightly light dose.

In yet another embodiment, the device can incorporate an alarm clock.

The embodiments mentioned above are intended for the purpose of illustration only and in no way are meant to the limit the manner in which the invention can be arranged.

A novel feature of the device is that the light therapy is delivered in variable daily dosages automatically calculated and controlled by a controller and designed to replicate the amount and intensity of illumination in naturally occurring illumination cycles like that of the lunar cycle.

Another novel feature of the device is that it allows the user to start her light therapy at the beginning of her existing cycle rather than waiting to synchronize the entrainment with the actual cycle of the moon or other natural illumination cycle. In this manner entrainment to, for example, a 29 day cycle occurs faster than if the user were required to entrain to the actual lunar cycle.

Yet another novel feature of the device is that it automatically indicates the dates of user's cycle with the highest probability of conception during intercourse without any further action by the user beyond the initial inputting of the start of her monthly cycle.

The device affords the user one or more of the following advantages. The primary advantages of the invention is that it improves the regularity and predictability of the user's cycle without any surgical or drug therapy. The treatment happens in the user's bedroom, or other darkened room while she is asleep or resting.

Another advantage the device can afford is that it does not interfere with the spontaneity or natural atmosphere of sexual intercourse. There is no need to perform any procedure with the device other than to actuate a user input control—once at the beginning of the user's cycle to activate the treatment regimen, and once at the time the user is going to bed to activate the nightly treatment after a specified delay period.

Another advantage of the device is that the improved regularity and predictability in the user's cycle that results from use of the device may be used to improve the probability of conception or reduce the probability of conception by synchronizing intercourse to dates that correspond to high or low probability of conception as indicated by the device.

Yet another advantage of the device is that it not only indicates which dates of the user's cycle have the highest probability of producing conception during intercourse, but it improves the accuracy of that prediction each month as the user's cycle becomes entrained to the simulated natural illumination cycle of the device.

Still another advantage of the unit is that it can compensate for the color scheme of any user's room by sensing the amount of light being reflected by the walls and ceilings and tuning the delivered illumination so that the correct dosage is provided in any room.

Still another advantage of the device is that the treatment cycle can be delayed each night until after the user has fallen asleep.

Still another advantage of the device is that it can be unplugged and taken with the user during periods of travel and plugged in again upon reaching the destination without losing track of the treatment cycle so that entrainment of the cycle is not interrupted during periods of travel or power outages.

Yet another advantage of the device is that it is very unobtrusive, having the appearance of a night light, so as not to reveal its purpose to casual observers or to call attention to itself.

Yet another advantage of the device is that it uses a glow indicator to enables the user to determine their predicted fertility status simply by looking at the device.

It is therefore the purpose of this invention to improve the regularity of a women's cycle by entraining it to a simulated natural illumination cycle and to indicate to the user which days of her cycle will result in the highest probability of conception during intercourse.

The present invention now will be described more fully hereinafter with reference to the accompanying drawings, which are intended to be read in conjunction with both this summary, the detailed description, and any preferred or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF DRAWINGS

FIG. 1 illustrates a top perspective of the device.

FIG. 2 illustrates a bottom, plan view of the device.

FIG. 3 illustrates a side elevation of the device.

FIG. 4 illustrates an end elevation of the device.

FIG. 5 illustrates a top perspective of the interior of the device including the controller.

FIG. 6 illustrates a top, plan view with upper housing removed.

FIG. 7 illustrates a bottom plan view lower housing removed.

FIG. 8 illustrates an exploded view.

FIG. 9 illustrates a lower perspective of circuitry and lightpipe.

FIG. 10 illustrates an exploded view of FIG. 9.

FIG. 11 illustrates a block diagram showing the relationship between internal components.

FIG. 12 illustrates a bottom perspective of an embodiment that does not comprise daily cycle progression indicator lights.

FIG. 13 illustrates a block diagram of an embodiment that does not comprise daily cycle progression indicator lights.

FIG. 14 illustrates a perspective of the wearable visor embodiment.

FIG. 15 is a chart showing the intensity of the light therapy by day of the cycle when using the preferred algorithm.

FIG. 16 is a chart showing the duration of the light therapy by day of the cycle when using the preferred algorithm.

FIG. 17 is a chart showing the total dosage of light by day of the cycle when using the preferred algorithm.

### BEST MODE FOR CARRYING OUT THE INVENTION

### Definitions

Entrainment—the gradual synchronization of a women's cycle to a schedule, other than the one that it would naturally follow, as a result of exposure to an external stimulus.
Dosage—intensity, duration and color of light delivered to the user during a single, light therapy session.
Treatment regimen—multiple dosages from the device over a period of therapy sessions.
Fertile days—the days during user's menstrual cycle when probability of conception during intercourse is increased; usually during the period of time starting 2-3 days before, to the day after ovulation
Subfertile days—all other days of the user's menstrual cycle that are not considered fertile days

### DETAILED DESCRIPTION

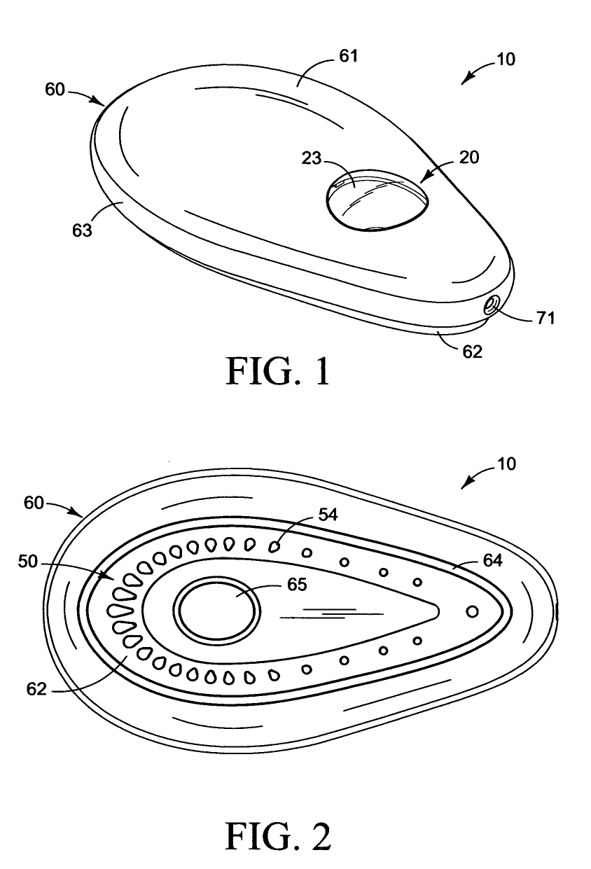

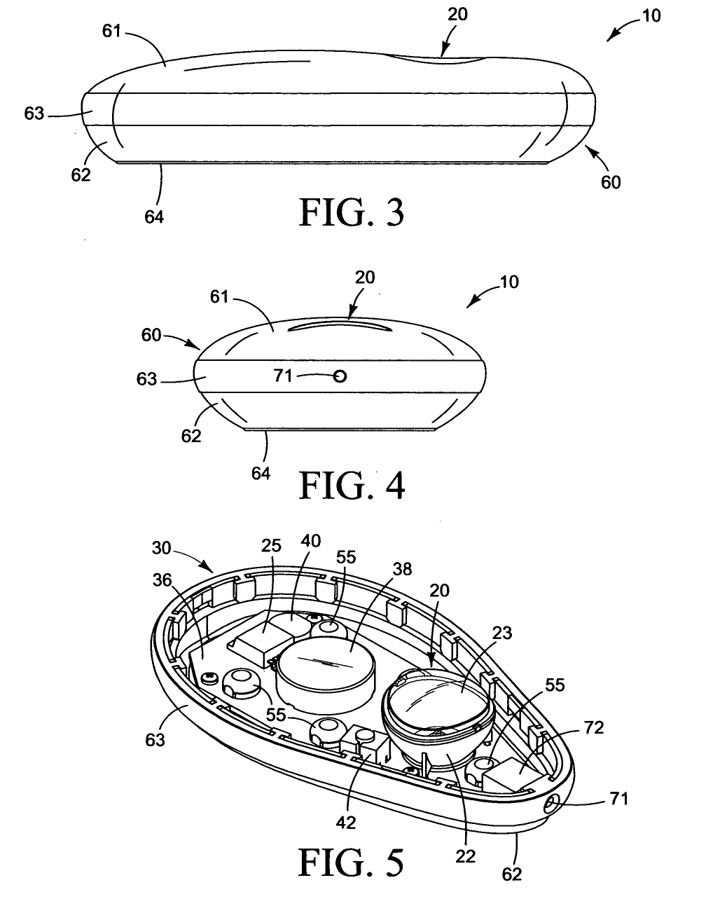

The present disclosure is directed to a luminescent device and method for regulating the cycle of a woman in order to improve the regularity of the cycle thereby facilitating natural family planning. As shown in FIGS. 1-5, an embodiment of the device 10 can comprise a light source 20 for producing the light to treat the user; a photo sensor 40 for detecting the amount of light reflected back to the unit; a controller 30 for determining and controlling the intensity, duration and schedule of light treatment and for determining what day of the user's cycle the user is in and which of the dates of the cycle the user is fertile; a housing 60 for containing the light source, sensor and controller; a plurality of indicator light sets 50, 55 for displaying how far along the woman is in her cycle and whether a particular day is associated with a high likelihood of conception; a plurality of input controls 38, 65 for accepting inputs from the user at the start; a means of supplying power 71 to the unit and circuitry 36 for connecting the various elements to each other.

Light Source

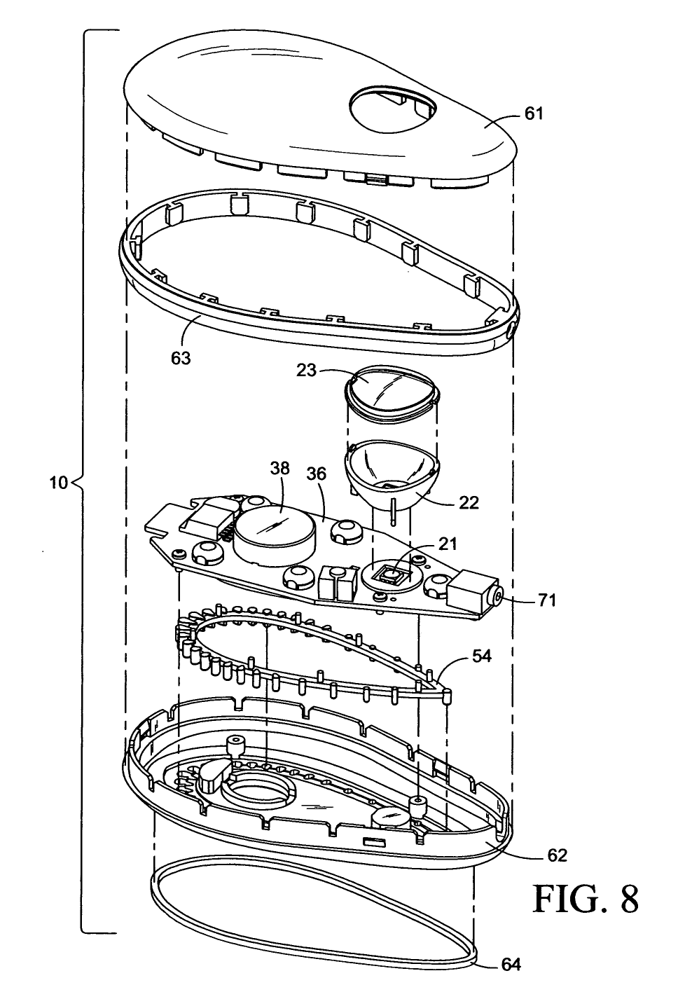

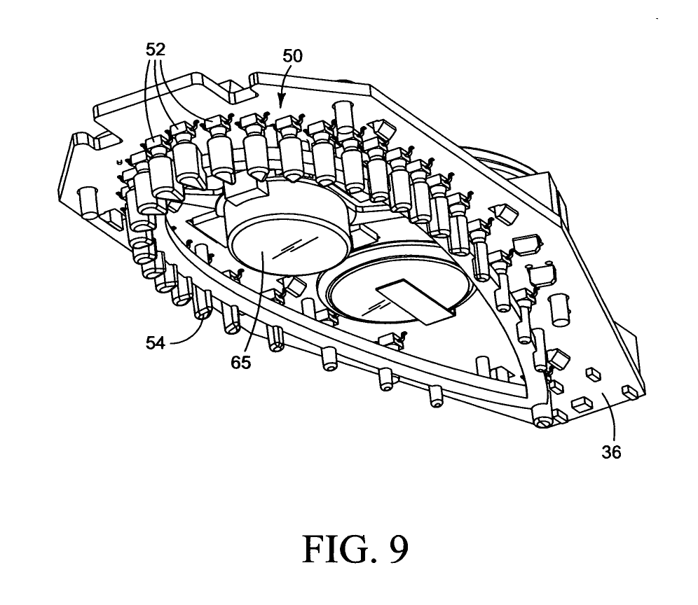

As shown in FIGS. 5-9, an embodiment of the light source 20 can comprise a lamp 21 for creating light in a specific color and intensity; a lamp driver 27 connected to the lamp which provides the power to the lamp according to desired treatment regimens, a reflector 22 surrounding the lamp which directs the rays of light upward and a lens 23 which further focuses the light in a collimated beam.

The lamp 21 can include, for example, incandescent bulbs, florescent bulbs or light emitting diodes (LEDs), preferably one or more light emitting diodes (LEDs). The lamp can produce light in a range of intensities and colors.

The reflector 22 comprises a conical, shiny surface of plastic or metal positioned below and around the lamp for purposes of directing the light upward and preventing it from diffusing laterally and shining too brightly into the face of the user.

The lens 23 comprises a clear, plastic dome covering the lamp and mates with the surface of the housing. It covers the lamp and reflector and prevents debris from being caught in the reflector. It also further focuses the light from the lamp in order to reduce diffusion of the light.

The lamp driver 27 comprises an integrated circuit and provides power to the lamp in accordance with signals sent to it from the controller 25. The driver 27 is connected to the lamp through circuitry.

The function of the light source is to emit the light that forms the basis of the entrainment therapy. It can be positioned on the top side of the housing so that it can direct its light upward toward the ceiling of the room where the device is positioned. It is connected to the controller through circuitry.

Controller

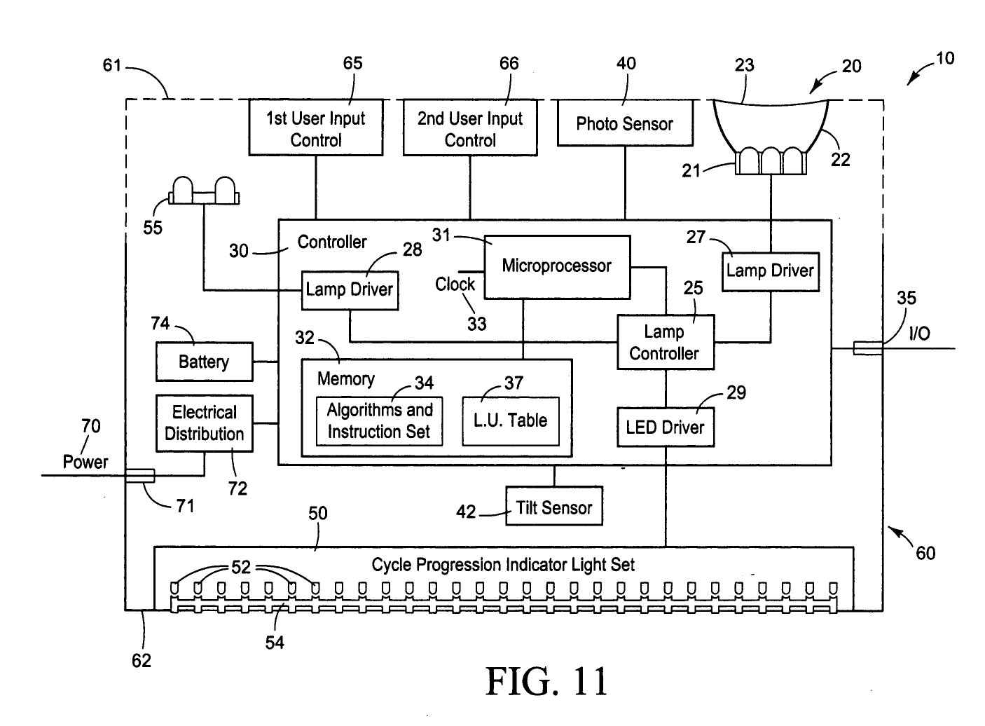

The controller 30, as shown in the embodiment of FIG. 11, comprises a clock 33, for example a crystal clock, a microprocessor 31, a memory 32, and an in-circuit programming connector, all of which are connected to each other and other elements of the device by circuitry.

The crystal clock 33 comprises an integrated circuit capable of keeping track of the elapsed time in days after a cycle start input signal is received and an elapsed time after a daily treatment session start signal is received.

The microprocessor 31 comprises an integrated circuit capable of receiving signals from the clock 33, a plurality of user input devices 65, 38, a photo sensor 40 and a memory 32. Based on these input signals the microprocessor performs calculations and manipulations of data that form the basis of the signals that it sends to the light source 20 and the indicator light sets 50, 55.

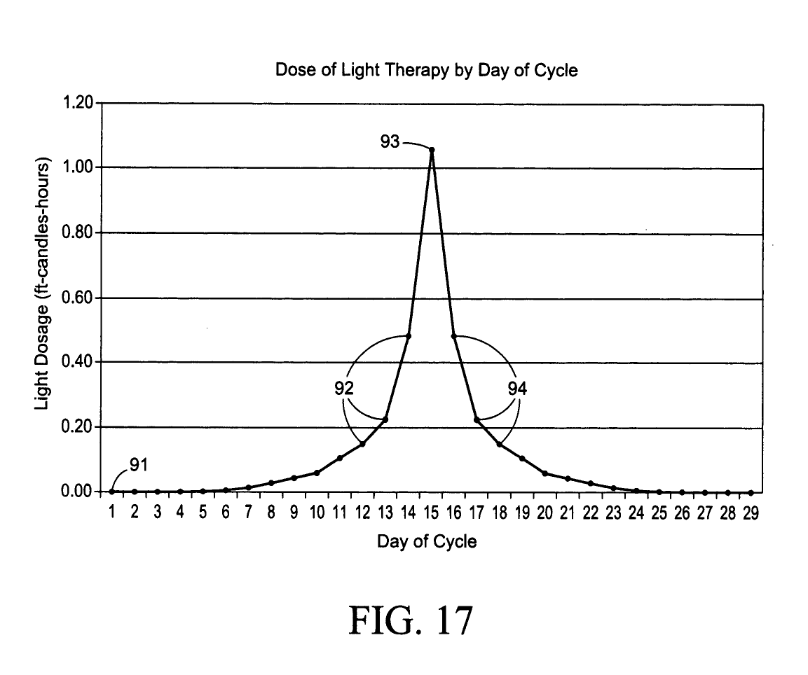

The memory 32 comprises a plurality of algorithms and instruction sets 34 and look-up tables 37 which operate in conjunction with the microprocessor 31 to create the treatment regimens and to predict the days of a cycle when the user is fertile. A first algorithm for controlling the light source, as shown in FIG. 17, defines a treatment regimen comprising: an initial dosage 91 of at least 0 ft candle-hours, said initial dosage occurring for at least one session of the treatment regimen; a series of sessions of incrementing dosages 92 following the sessions at the initial dose level 91, wherein the increments are greater than 0 and continue for at least one session of the treatment regimen; a maximum dosage 93 following the series of incrementing dosages wherein the maximum dosage occurs for at least one session of the treatment regimen; a series of sessions of decrementing dosages 94 following the maximum dosage wherein the decrements are greater than 0 and the series of decrementing sessions lasts until the dose level equals the initial dose level of the treatment regimen.

A second algorithm is based on a look-up table 27 which produces an incrementally increasing light dosage for the first half of a 29 session treatment regimen and an incrementally decreasing dosage for the second half. The incremental changes from day to day are not necessarily uniform in size and the maximum dosage can be shifted earlier or later in the treatment regimen. Both algorithms indicate the same set of days during each cycle as the user's fertile days.

Yet another algorithm uses the same treatment regimen for controlling the light source as the first or second algorithm but uses an adaptive method for indicating the dates of the user's cycle that represent fertile days. This algorithm takes into account the difference between the predicted start date of the current cycle and the actual start date of that cycle indicated by the user through an input control 65 actuated on the actual cycle start date. The difference between the predicted and actual start date is incorporated as feedback and adjusts the predicted dates for the current cycle with the expectation that the difference between predicted and actual will decrease over time as entrainment becomes more complete.

Still another algorithm can produce a treatment regimen that follows a step function whereby the dosage changes from zero to full intensity and remains at that intensity for each subsequent treatment session until returning to and remaining at zero for the remainder of the treatment regimen.

The in-circuit programming connector 35 comprises a port connected to the microprocessor 31 by circuitry 36, which mates with standard hardware such as USB or RS292 connections to enable data, algorithms and instruction sets 34 to be loaded into the microprocessor.

The purpose of the controller 30 is to control functions of the device by receiving signals from user input devices 38, 65 and sensors 40, sending signals to the light source 20 and indicator lights 50, 55, and storing and retrieving data and programming in order to control the light source 20 so that it produces light in accordance with the desired treatment regimen. It is located inside the housing 60 and connected to the light source 20, sensor 40, user input controls 38, 65 and indicator light sets 50, 55 through circuitry.

Sensor

The photo sensor 40 comprises a light intensity measuring device and measures the intensity of the light reflected back to the device from the surrounding environment and sends a signal to the controller 30 corresponding to the intensity of the reflected light so the controller can adjust the signal sent to the light source so that the desired intensity of reflected light is achieved.

In an embodiment the photo sensor 40 can be positioned in an upward facing surface of the housing and is connected to the controller by circuitry.

Housing

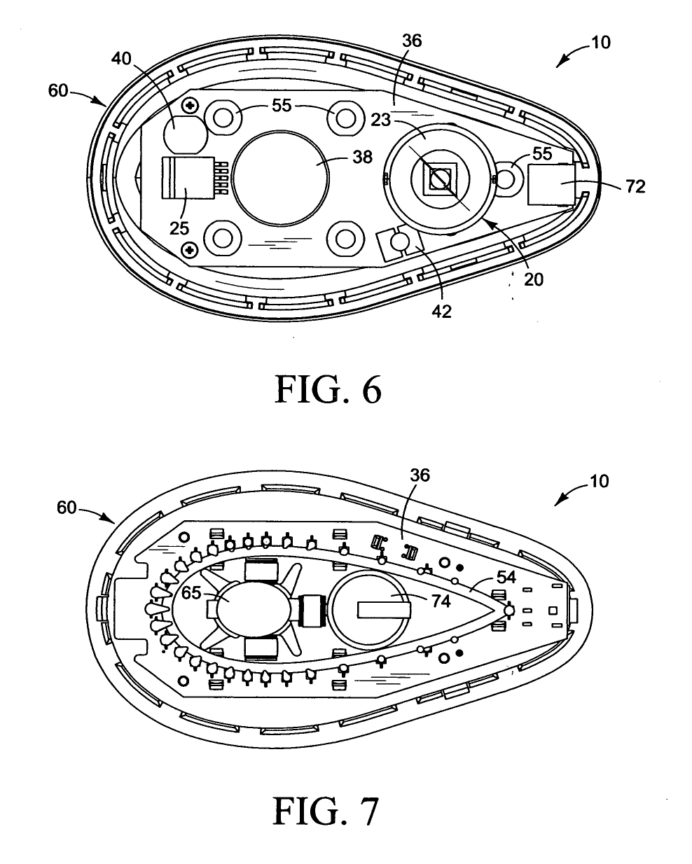

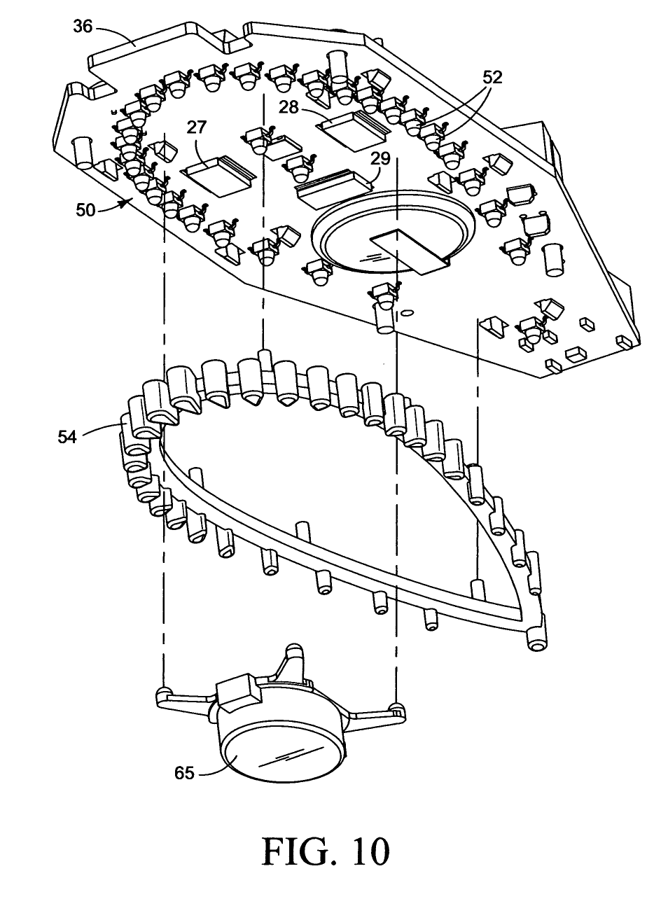

As shown in the embodiments of FIGS. 6-11, the housing 60 comprises a top shell 61, a base 62, a spacer 63 and a foot 64. The housing 60 is large enough to hold the other elements and to provide enough surface area to allow for the clear display of the indicator lights 50, 55 and the input controls 38, 65, while being small enough to fit on a typical nightstand.

The top shell 61 can comprise a dome shaped shell and can be made of semi-transparent material such as injection molded plastic and is designed to allow light from probability indicator light sets 55 to pass through it thereby creating a colored glow that indicates fertility status in an easily visible manner. The top shell 61 also mates with and surrounds the lens 23 of the light source 20 in order to allow the therapy light to be transmitted upward through the top shell 61.

The base 62 can comprise a hemispheric, open vessel with a circumference identical in size and shape to that of the top shell 61. It can be made of a variety of materials such as metal or plastic, preferably metal to enhance heat transfer from the components inside the base to the ambient surrounding the base. The base 62 provides the structural rigidity to the device and provides a surface to which the other elements of the device are attached.

The spacer 63 fits between the tops shell and the base and is made of a compliant material to enhance tactile sensation and improve grip.

The foot 64 can be made of a compliant, soft material such as rubber or felt. It attaches to the underside of the base 62 by adhesive or press fitting into a channel in the base 62. The foot 64 provides a non skid material on the underside of the device so that it does not wobble on or slide off of surfaces.

Cycle Progression Indicator Light Set

As shown in the embodiments of FIGS. 6-11, the first indicator light set is the cycle progression indicator light set 50 and comprises a lamp controller 25 and driver 29, a plurality of individual lights 52, and a light pipe 54. The individual lights range in number from 28-31, preferably 29, one for every day of the female cycle to which the device 10 is entraining the user. The individual lights 52 include, for example, light emitting diodes (LEDs) to create the light. The lights 52 are in contact with a light pipe 54 which transmits the light created by the individual lights 52 to the surface of the base 62 from which the light-pipe 54 protrudes slightly. The indicator lights 52 may be a plurality of colors, shapes or sizes so that lights representing certain days of the cycle, in particular the fertile days, can be distinguished from the other lights. The individual lights 52 of the cycle progression indicator light set 50 are connected to the light driver 29 and lamp controller 25 by circuitry and light up one at a time to indicate the day of the user's cycle and to provide an indication whether the user is likely to be fertile on an indicated day.

Probability Indicator Light Set

The second indicator light set is the probability indicator light set 55 and comprises a plurality of lights 56, and a driver 28. The lights can be made from any small light bulb, but preferably from light emitting diodes (LEDs) capable of producing light in a variety of colors and intensities. The purpose of this indicator light set is to provide back-lighting of the semi-transparent top shell 61 of the housing in order to provide a quick indication to the user of her fertility on a given day so that the user does not have to consult the cycle progression light set 50 at every occasion. This is accomplished by illuminating the top shell 61 with a highly configurable combination of light patterns, colors and intensities to indicate fertility status or related information. The probability indicator light set 55 is positioned under the top shell 61 of the housing 60 so that the light it emits back-lights the top shell 61. The light set 55 is connected to the light driver 28 and light controller 25 via circuitry which themselves are further connected to the main controller 30 via additional circuitry.

Input Controls

The input controls comprise a cycle start input control 65 and a session start input control 38. The cycle start input control 65 can be located on the underside of the base 62 of the housing 60. The session start input control 38 can be located beneath the surface of the top shell 61 of the housing 60. The cycle start input control 65 is actuated by the user on the first day of the user's cycle and can be actuated by pressing it. The session start input control 38 is actuated by the user when the user desires the daily treatment session to begin, usually at the time the user goes to bed. The session start input control 38 can be actuated by pressing it or by configuring the control to respond to the sound created by the user tapping the top shell 61 of the housing 60 with the user's finger.

Power Unit

The power unit 70 comprises a power receptacle 71, an electrical distribution circuit 72 to accept and transform AC power and transmit it to the other elements, an external power detector circuit, and back-up batteries 74 to maintain power to the unit during travel and power outages. In an alternative embodiment the AC circuitry is replaced with a DC only supply.

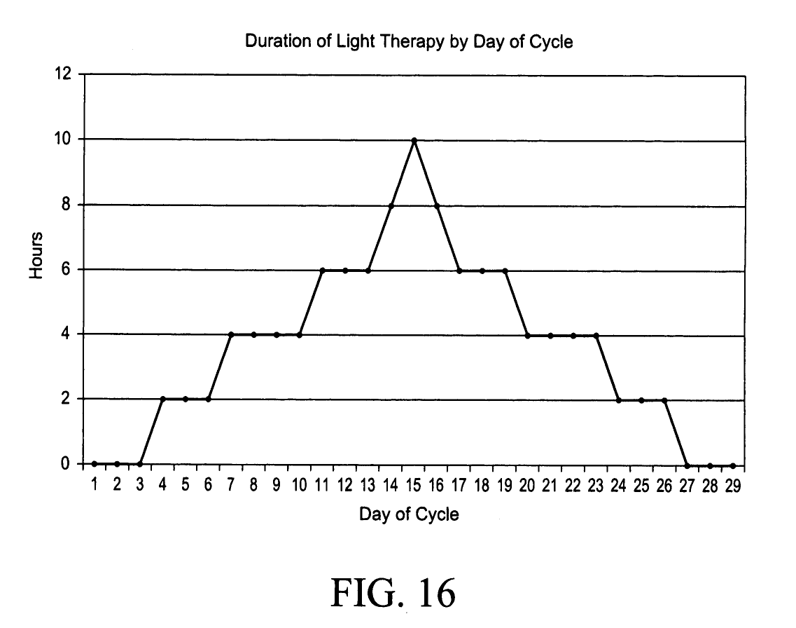

In an embodiment the device 10 emits light designed to simulate natural moonlight. The light is created by a light emitting diode capable of producing light in intensities ranging from 0.01 to 10 foot candles, such as about 0.1 foot candle, and in a range of multi-spectral temperatures (color) between 2800K and 4800K, such as about 4100K, which is the color of natural moonlight. The light source 20 is contained inside the housing 60 which is sized so that it will fit conveniently on a bedside nightstand or similar piece of bedroom furniture. The controller 30 controls the treatment regimen by tracking the time elapsed since the start of the users cycle and by calculating and controlling the nightly dosage of simulated moonlight in any suitable dosage profile, for example dosages in quantities ranging from zero to a maximum dose at some point of the cycle and thereafter trailing off to zero again at the end of the cycle, as shown in FIG. 17. The dosages are determined by the controller 30 and can be derived from algorithms 34 and look-up tables 37 contained in the memory which provides duration, intensity and ramp up and ramp down periods for each dosage of the treatment regimen. For example as shown in FIG. 16, the first 3 sessions can provide no light exposure, the next three sessions 2 hours each, the next four sessions 4 hours each, the next three sessions 6 hours each and the next session 8 hours and the next session 10 hours. The dosages can then be stepped down in subsequent sessions in the same manner in which they were increased, ending with three sessions of no light exposure. The intensity of the light provided during each treatment session can be ramped up and ramped down to a peak intensity as well. The information to create the treatment session is processed in the microprocessor 31 and sent as a signal to the lamp driver 27 through circuitry thereby allowing the driver 27 to control the output of the lamp 21 in accordance with the dosage determined by the controller 30. The housing 60 also contains a photo sensor 40 which measures the intensity of the reflected light and adjusts the output of the lamp driver 27 so that the reflected light falls within an intensity range specified by the controller for each dosage. The housing 60 further contains the input controls 38, 65 including a manually actuated control 65 for indicating the start of the users cycle and a second control 38 to indicate that the user is going to bed and wishes for the treatment regiment to commence after a specified delay time, such a control, for example, being an audible tap device which detects the sound created when the user touches the top of the device. The inputs from these devices are transmitted by circuitry 36 to the controller 30 in order to activate the monthly treatment regimen, the nightly dosage calculations and the nightly start time for the dosage as well as the algorithms 34 for predicting and indicating fertile days of the cycle. The housing 60 can also contain a cycle progression indicator light set 50 in the form of 28-31, preferably 29, smaller LEDs arranged about the underside of the device 10 in a ring or horse-shoe arrangement, each LED corresponding to a day of the ideal lunar-entrained female cycle. The LEDs 52 are also connected to the controller 30 and activate one by one on each successive day of the cycle so the user can tell with a quick glance which day of the cycle she is in. Those LEDs corresponding to the fertile days of the users cycle can be larger than the lights of the other days in order to indicate to the user that the days associated with the larger lights are the most fruitful days for conception-producing intercourse. The LEDs 52 can be activated by a tilt sensor which enables power to flow to the lights when the device 10 is picked up by the user and turned over. Otherwise, these lights 52 can remain off. In addition to the cycle progression indicator light set 50 on the underside of the device 10 the top shell 61 of the device is capable of back-lit illumination from another set of LEDs 55 so that the entire top shell 61 of the housing 60 will glow with a specified color during the fertile days of the users cycle. This second indicator method obviates the need for the user to look at the underside of the device to check the precise day of her cycle while still providing an unobtrusive reminder that the user is in a fertile state. The device 10 also comprises a user activated nightlight that enables the user to see during the night without negatively affecting the nightly light treatment. The device 10 further comprises an AC power receptacle 71 and cord which enables the device to plug into normal household 120V AC outlet. In addition the device 10 contains a battery 74 capable of maintaining the memory 32 of the controller 30 in a refreshed state when the device 10 is unplugged for travel or during power outages.

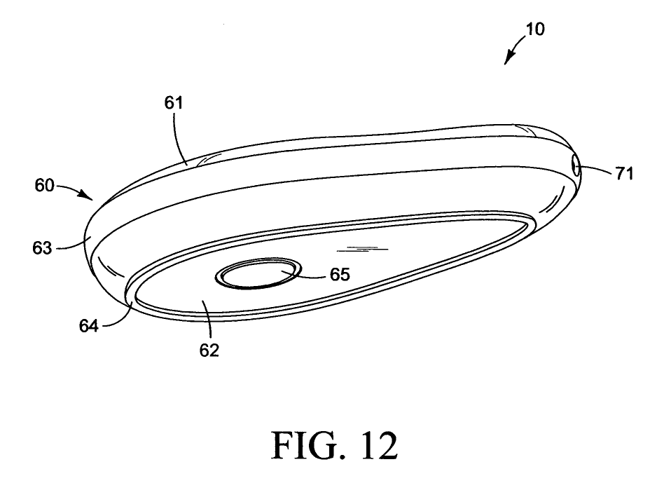

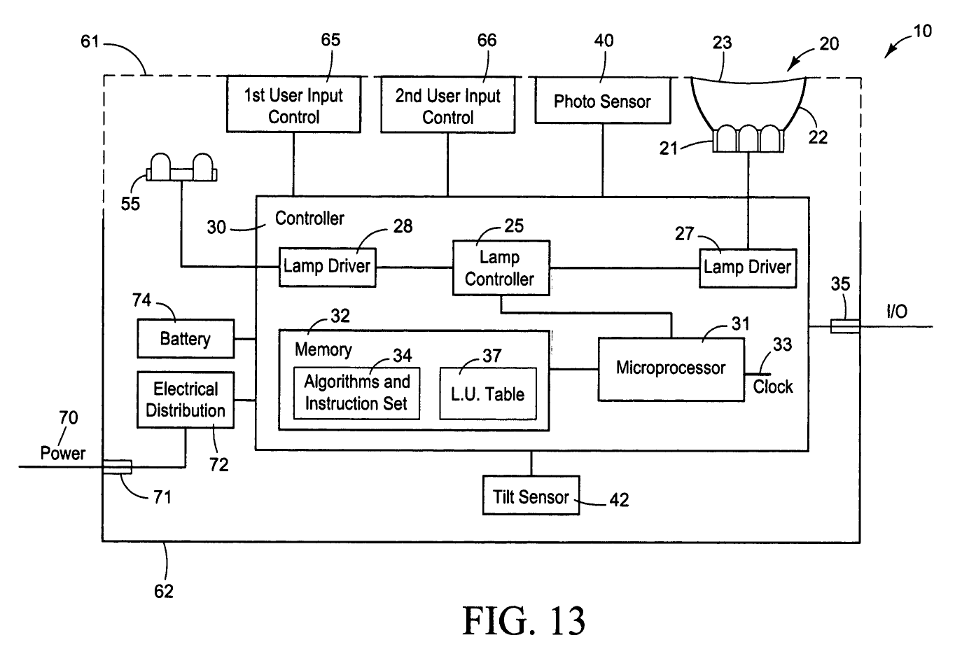

In an embodiment, as shown in FIGS. 12-13, the device is as described above except that the daily cycle progression indicator light set is not present and only the probability indicator light set 55 is available to indicate conception probability.

In an embodiment, the lights of the cycle progression indicator light set 50 which are distinguishable from the remainder of the lights 52 for the purpose of indicating the predicted fertile dates of the users cycle are further distinguishable to indicate which days of the fertile days offer an improved chance of conceiving a male offspring and which days offer an improved chance or conceiving a female offspring. The designation of certain days within the fertile period as offering enhanced sex selection probability for one sex or the other can be based on any suitable method such as for example the Shettle's method which postulates that sperm which result in male offspring swim faster, but are less robust, than sperm which produce female offspring. Hence fertilization of the egg by male-producing sperm is more likely if intercourse occurs very close to ovulation. Sperm which result in female offspring swim slower but are more robust. Hence fertilization of the egg by those sperm is more likely if intercourse occurs several days prior to ovulation.

In an embodiment the device includes no light source for treatment and only includes the housing 60, controller 30, indicator light sets 50, 55 cycle start input control 65 and power unit 70. Therefore it provides no light therapy and only indicates the days of the user's cycle that the user is in and the days of the user's cycle offering increased likelihood of conception during intercourse. This embodiment can also be configured with the adaptive algorithm which modifies the days of the user's cycle indicated to be of increased likelihood of conception based on the difference between the predicted start of the user's menstrual cycle and the actual start date indicated to the device by the user.

In an embodiment the light source 20 of the device 10 includes blue LEDs emitting light in a narrow range of wavelengths from about 465 to about 470 nm instead of the multi-spectral light produced by LEDs with temperature ranges from 2800K to 4800K, the blue light being particularly useful in treatment of circadian rhythm disorders.

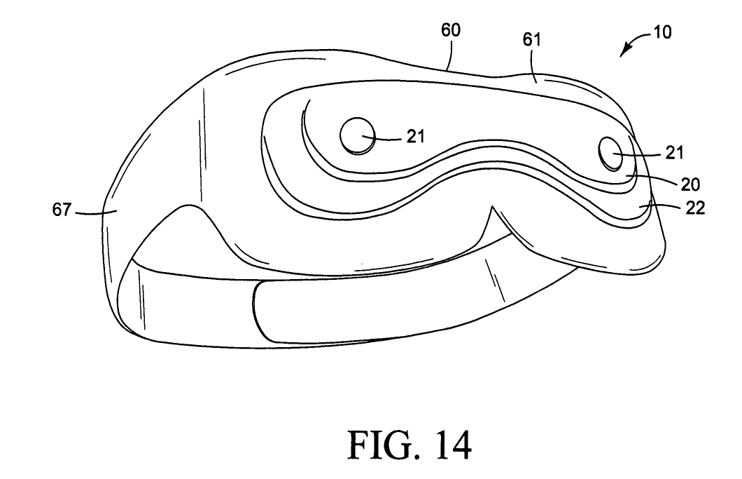

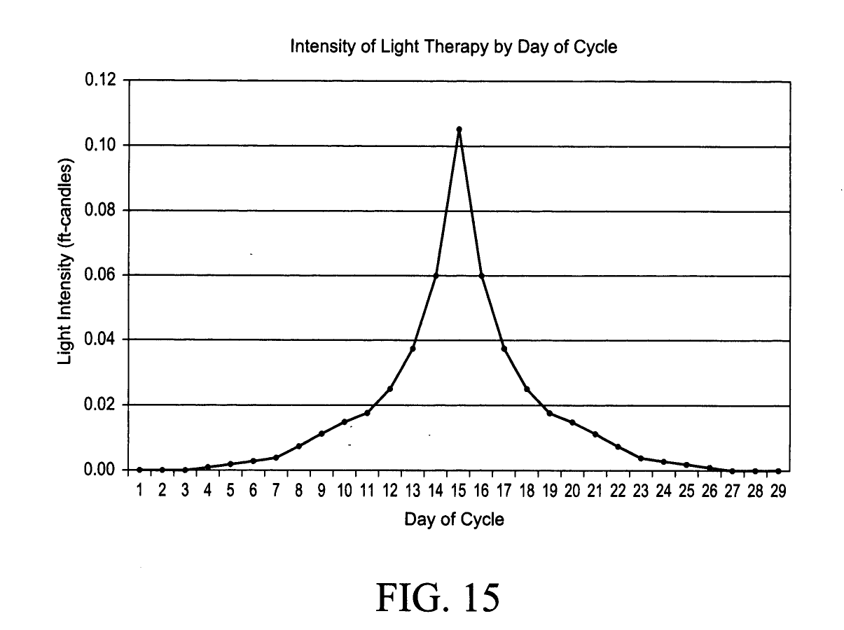

In still another embodiment, as shown in FIG. 14, the device 10 can be a wearable apparatus in the form of a visor. It can comprises a frame 67, which allows the unit to be worn over the users eyes, a housing 60, a light source 20, a controller, a probability indicator light set 55 and a power unit 70. The housing 60 can comprise a semi transparent top shell 61 to allow the display of the probability indicator light set 55 as in the embodiment of the bed-side unit. The light source contains a lamp 21 and a reflector 22. The frame 67 can be made from any light-weight, high-strength material such as injection molded plastic. The reflector 22 can be made of a plastic reflective material or a semitransparent material capable of diffusing the light from the lamp throughout the material. In this embodiment power can be provided by a DC supply with replaceable or rechargeable batteries.

In still another embodiment the device can include elements associated with common bedside accessories such as night lights and alarm clocks.

While the present device has been described above in terms of specific embodiments, it is to be understood that the device is not limited to these disclosed embodiments. Many modifications and other embodiments of the device will occur to those skilled in the art to which this invention pertains. These alternative embodiments are intended to be and are covered by both this disclosure and the appended claims. It is indeed intended that the scope of the disclosure should be determined by proper interpretation and construction of the appended claims and their legal equivalents, as understood by those of skill in the art relying upon the disclosure in this specification and the attached drawings.

### INDUSTRIAL APPLICABILITY

The device 10 can be used by placing it on a surface in a room and plugging it in to an AC wall receptacle and then turning it on. The room must be capable of being darkened during the therapy sessions, which are intended to occur at night while the user, typically a female treatment subject, sleeps, although the therapy sessions can be at any time of the day. On the day that the user begins her cycle a first input control 65 is actuated on the device 10 which signals the controller that the user's cycle has begun. This triggers the internal clock 33 of the device which then begins to track the elapsed days of the user's cycle. Then, each night before going to bed, the user actuates a second input control 38 indicating her readiness to begin the light therapy. After a specified delay period, which affords the user the opportunity to go to sleep before exposure to the light, the therapy begins in the form of light emitted from the light source 20 and reflected off the ceiling onto the subject's open or closed, but uncovered eyes. Each night as the therapy is repeated, the device emits a specified dosage of light in an intensity and duration that approximates that which would be emitted by the moon on the corresponding night of its lunar cycle with the dosages increasing steadily towards a maximum intensity and duration on day 15 of the simulated cycle and then decreasing steadily to day 29. Furthermore, during each nightly treatment session that calls for light exposure, the light is programmed to turn on and off gradually to further simulate lunar illumination and to prevent waking the user. The illumination regimen created by the device could also simulate other natural or artificial illumination cycles besides that of the moon.

During the course of the treatment regimen the user consults the indicator lights 50, 55 of the device 10, at any time when the user is not receiving light therapy, in order to determine what day of her cycle the user is in and to gauge the likelihood of conception during intercourse on that day. This is accomplished either by looking at the cycle progression indicator light set 50 which illuminates a unique light on each day of the cycle and provides distinguishing characteristics to the lights associated with days of predicted fertility, or by looking at the color coding of the probability indicator light set 55 on the top side of the device which glows in a distinctive color and pattern when the user is in the days of her cycle in which she is predicted to be fertile. Where the possibility of influencing sex selection is also desired, the user consults the additional distinguishing characteristics of the lights indicating predicted fertile days to determine which days within the fertile period offer improved probabilities of conception resulting in a particular sex of the fetus. Once the cycle has ended, the user provides no further inputs to the device 10 until her cycle begins again in which case the input control that signals cycle commencement is activated again. In the event the user must travel or move to another location during the therapy regimen she unplugs the device 10 and takes it with her and installs it at her new location. The internal battery 73 and clock 33 keep track of the cycle status while unplugged and resume the treatment regimen at its proper day of the cycle when plugged back in.

In the alternate, visor embodiment the device 10 is worn by the user while she sleeps and the light therapy is delivered directly to the user's face without reflection off the surrounding room, so that others sharing the room with the user are not disturbed by the reflected light. The user actuates an input control at the onset of her cycle. Then, when ready to begin her therapy, she actuates a second input control, dons the visor and goes to bed. When the user awakens in the morning, the user removes the visor and, if necessary, plugs it into its charging unit so that the unit remains charged. As with the preferred embodiment, the user consults the indicator lights on the housing at any time when it is not being worn in order to determine what day of her cycle she is in and what the likelihood of conception during intercourse is on that day.

Although intended primarily for female women, the device is not restricted to that application and embodiments of it can be used for female animals such as livestock and fish.

1. A device for influencing and predicting a woman's female cycle comprising:
a means for delivering a light-therapy regimen to influence the cycle; and
a plurality of indicators for indicating the predicted likelihood of conception on a given day of the cycle, said indicators being synchronized with the means for delivering a light-therapy regimen.

2. The device of claim 1, wherein the means for delivering a light-therapy regimen comprises a lamp (21) capable of producing blue light at a range of wavelengths ranging from about 465 to about 460 nm.

3. The device of claim 1, wherein the means for delivering a light-therapy regimen comprises a lamp (21) capable of producing multi-spectral light in a color ranging from about 2800K to about 4800K, and an intensity ranging from about 0.01 to about 10 lumens.

4. (canceled)

5. The device of claim 1, wherein at least one indicator comprises a means of predicting the probability of conception during the cycle.

6. The device of claim 5, wherein the at least one indicator comprises:
a plurality of indicator lights (50), one for each day of the cycle, the lights being connected to the means for delivering a light-therapy regimen and configured to light to apprise the woman of which day of the cycle the woman is in; and
a subset of the indicator lights being distinguishable from the remaining indicator lights, said subset configured to indicate the relative likelihood of conception on a given day of the cycle.

7. The device of claim 6, further comprising:
a clock (33) for keeping track of the day and time;
a microprocessor (31), connected to the clock, for processing inputs and making calculations related to the light-therapy regimen;
a memory unit (33), connected to the microprocessor, comprising:
an algorithm (34); and
a look-up table (37);
a means for determining a dosage of light-therapy by referencing the algorithms and look up tables stored in the memory unit;
a means for sending a signal to the means for delivering a light-therapy regimen to deliver a predetermined dosage of light at a predetermined time;
a means for determining which day of the cycle the woman is in based on inputs indicating the start date of the woman's cycle and signals from the clock;
a means for determining which day of the cycle the woman is likely to be fertile based on information from the memory unit; and,
a means for controlling the plurality of visual indicators.

8. The device of claim 7, wherein the light-therapy regimen comprises:
an initial dosage of at least 0 ft candle lumens, said initial dosage occurring for at least one session of the treatment regimen;
a series of sessions of incrementing dosages following the sessions at the initial dose level, wherein the increments are greater than 0 and continue for at least one session of the treatment regimen;
maximum dosage following the series of incrementing dosages wherein the maximum dosage occurs for at least one session of the treatment regimen; and
a series of sessions of decrementing dosages following the maximum dosage wherein the decrements are greater than 0 and the series of decrementing sessions lasts until the dose level equals the initial dose level of the treatment regimen.

9. The device of claim 8, further comprising a light sensor and a means for modulating the intensity of the light-therapy regimen based on input from the light sensor.

10. The device of claim 9, further comprising an input control (38) for detecting the woman's tactile interface with the device, and for sending a signal to the means for delivering a light-therapy regimen to control a treatment session.

11. The device of claim 10, further comprising a red-colored night light.

12. The device of claim 11, further comprising an alarm clock.

13. The device of claim 7, wherein the means for delivering a light-therapy regimen further comprises a means for determining the days of the woman's cycle that are designated as fertile days by recording the difference in days between the receipt of the signal from the means for delivering a light-therapy regimen indicating the start of the woman's cycle and the date stored in the look-up table (37) of the memory unit (32) as the predicted start of the cycle and incorporating that difference between the dates into the means for determining which day of the woman's cycle the woman is likely to be fertile.

14. A device for predicting a user's female cycle comprising:
a controller (30) for predicting the days of the user's cycle the user is likely to be fertile, the controller comprising a memory (32) containing a look-up table (37);
an input control (65) connected to the controller, the input control configured to send signals to the controller in order to indicate the start of the user's cycle;
a plurality of indicators (50, 55), connected to the controller, to indicate during which days of the user's cycle the user is likely to be fertile based on signals received from the controller; and
a power source for providing power to the device.

15. The device of claim 14, wherein the controller (30) further comprises a means for determining the days of the user's cycle that are designated as fertile days by recording the difference in days between the receipt of the signal from the input control (65) indicating the start of the user's cycle and the date stored in the look-up table (37) of the memory (32) as the predicted start of the cycle and incorporating that difference between the dates into the means for determining which day of the user's cycle the user is likely to be fertile.

16. The device of claim 14, wherein one of the indicators comprises:
a plurality of indicator lights (50), one for each day of the user's menstrual cycle, the lights being connected to the controller (30) and configured to light to apprise the user of which day of the cycle the user is in; and,
a subset of the indicator lights being distinguishable from the remaining indicator lights, said subset configured to indicate the relative likelihood of conception on a given day of the cycle.

17. The device of claim 1, wherein the means for delivering a light-therapy regimen defines a treatment regimen comprises:
an initial dosage (91) of at least 0 ft candle lumens, said initial dosage occurring for at least one session of the treatment regimen;
a series of sessions of incrementing dosages (92) following the sessions at the initial dose level, wherein the increments are greater than 0 and continue for at least one session of the treatment regimen;
a maximum dosage (93) following the series of incrementing dosages wherein the maximum dosage occurs for at least one session of the treatment regimen; and
a series of sessions of decrementing dosages (94) following the maximum dosage (93) wherein the decrements are greater than 0 and the series of decrementing sessions lasts until the dose level equals the initial dose level of the treatment regimen.

18. (canceled)

19. A method for influencing a user's likelihood of conception during intercourse comprising the steps of:
providing a device for influencing and predicting a female cycle in a woman according to claim 1;
providing power to the device;
indicating to the device the onset of a woman's menses by actuating a first input control (65) on the device;
providing a predetermined dosage of light by regulating the means for delivering a light-therapy regimen;
indicating to the user which dates of the woman's cycle the user is likely to be fertile; and,
instructing the woman to engage in intercourse on days whose likelihood of conception correspond to the woman's family planning objectives.

20. A method as in claim 19, wherein the providing step comprises exposing the woman to zero hours of emitted light for the first 7 treatment sessions after the onset of menses, an increasing predetermined dosage of emitted light for the next 7 sessions, a constant predetermined dosage of emitted light for the next 2 sessions, a decreasing predetermined dosage of emitted light for the next 7 sessions, and zero hours of emitted light for the next 6 sessions.

21. A method as in claim 19, wherein the instructing step further comprises:
asking the woman which gender of child the woman prefers to conceive,
indicating to the woman which days of the cycle favor conception of a particular gender; and,
instructing the woman to have intercourse on the dates indicated to favor conception of the gender preferred by the woman.
